(19) United States
(12) Patent Application Publication
(10) Pub. No.: US 2006/0172320 A1
(43) Pub. Date: Aug. 3, 2006

(54) Modular aptametric sensors without covalently attached fluorophores

(76) Inventor: Milan N. Stojanovic, Fort Lee, NJ (US)

Correspondence Address: John P. White;Cooper & Dunham LLP, 1185 Avenue of the Americas, New York, NY, 10036, US

(21) Appl. No.: 11/180,082

(22) Filed: Jul. 13, 2005

Related U.S. Application Data
(60) Provisional application No. 60/588,150, filed on Jul. 13, 2004.

(51) Int. Cl. C12Q 1/68

(52) U.S. Cl. 435/6

(57) ABSTRACT

Modular aptameric sensors, transduce recognition events into fluorescence changes through allosteric regulation of non-covalent interactions with a fluorophore. These sensors consist of: (a) a reporting domain, which signals the binding event of an analyte through binding to a fluorophore; (b) a recognition domain, which binds the analyte; and (c) a communication module, which serves as a conduit between recognition and signaling domains. We tested recognition regions specific for ATP, FMN and theophylline in combinations with malachite green binding aptamer as a signaling domain. In each case, we obtained a functional sensor capable of responding to an increase in analyte concentration with an increase in fluorescence. Similar constructs that consist only of natural RNA could be expressed in cells and used as sensors for intracellular imaging.

### STATEMENT REGARDING FEDERALLY SPONSORED RESEARCH OR DEVELOPMENT

The sensor work described herein is supported by the NIH (NIBIB, RO1 EB00675-1) and the NSF (Biophotonics Grant, BES-03). Work on the recognition-triggered small molecule release (and binding) is funded by the NASA (NAS2-02039).

### BACKGROUND OF THE INVENTION

Throughout this application, various publications are referenced to by numbers. Full citations may be found at the end of the specification immediately preceding the claims. The disclosures of these publications in the entireties are hereby incorporated by reference into this application in order to more fully describe the state of the art to those skilled therein as of the date of the invention described and claimed herein.

Several groups (1-5), including ours (6-9), recently reported successful approaches to fluorescent aptameric sensors for small molecules and proteins. However, none of these approaches are readily adaptable to intracellular imaging applications. In particular, the reported methods depend on labeled or unnatural DNA or RNA molecules. Therefore, the sensors require exogenous delivery, in contrast to fluorescent proteins that can be expressed in cells (10). Previously described malachite green RNA aptamer (11) (MGA, FIG. 1) increases the quantum yield of this dye up to 2000-fold upon binding (12)

Modular design has previously been applied to achieve the allosteric regulation of nucleic acid catalysts. (14-17) While allosteric aptamers binding dyes and small molecules have been isolated through a selection-and-amplification procedure earlier (13), the lack of effective readout hindered practical applications of similar systems. We have recently achieved calorimetric readout using isosteric antagonistic binding between a dye and cocaine (7).

### SUMMARY OF THE INVENTION

We tested modular aptameric constructs combining a malachite green RNA aptamer as a “signaling domain” with other aptamers as “recognition domains”. We obtained a series of allosteric (13) aptamers, containing no chemical modifications and showing fluorescence changes upon binding simultaneously malachite green (MG) and target analytes, ATP, flavin mononucleotide phosphate sodium (FMN), and theophylline (TH). FMN and TH sensors consist only of RNA and, thus, represent the proof-of-principle of expressable aptameric sensors.

However, this system had little potential for general intracellular applications. Free malachite green dye has only negligible fluorescence. This feature kindled our interest in the allosteric regulation of binding events in nucleic-acid aptamers. In particular, for the first time we could test our ability to couple binding of analytes and dyes in specific and separate binding pockets, and with concomitant analyte-dependent change in fluorescence. Up to now, and unlike with proteins, oligonucleotides that spontaneously form fluorophores have not been discovered. With the development of this new system, we could express the allosteric aptamer, add the dye to a media, and follow the formation of the fluorescent complex. Our longterm plan is to expand this ability to regulate fluorescence of the non-covalent complexes through allosteric effects to intracellular and, eventually, to in vivo applications.

According to the invention, a method of detecting whether a specific compound is present in a test solution is provided comprising: (a) providing a composition comprising an oligonucleotide comprising a recognition portion, a reporting portion, and a double stranded stem portion which connects the reporting portion to the recognition portion, wherein a fluorescent dye is bound to the reporting portion; (b) contacting the composition with a control solution; (c) quantitating the fluorescence of the composition in contact with the control solution; (d) contacting the composition with the test solution under conditions which permit any of the specific compound present in the test solution to bind to the recognition portion and thereby alter the fluorescence of the composition without displacing the fluorescent dye from the reporting portion; and (e) quantitating the fluorescence of the composition in contact with the test solution, wherein a difference between the fluorescence quantitated in step (c) and step (e) indicates that the specific compound is present in the test solution.

According to the invention, a method of detecting ATP in a test solution is provided comprising: (a) providing a composition comprising an oligonucleotide which comprises the following structure, wherein M is a malachite green dye: 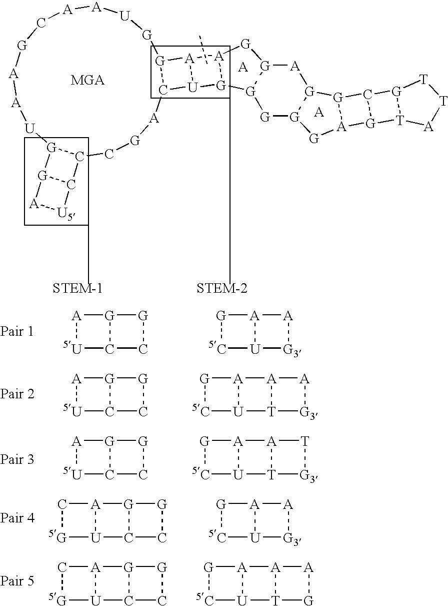
(b) contacting the composition with a control solution; (c) quantitating the fluorescence of the composition in contact with the control solution; (d) contacting the composition with the test solution under conditions which permit any of the ATP present in the test solution to bind to the ATP-recognition portion and thereby alter the fluorescence of the composition without displacing the fluorescent dye from the fluorescent module; and (e) quantitating the fluorescence of the composition in contact with the test solution,
wherein a difference between the fluorescence quantitated in step (c) and step (e) indicates that the ATP is present in the test solution.

According to the present invention a method of detecting theophylline in a test solution is provided comprising: (a) providing a composition comprising an oligonucleotide which comprises the following structure, wherein M is a malachite green dye: 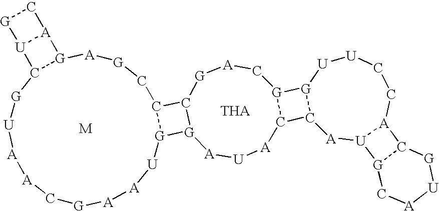
(b) contacting the composition with a control solution;(c) quantitating the fluorescence of the composition in contact with the control solution;(d) contacting the composition with the test solution under conditions which permit any of the theophylline present in the test solution to bind to the theophylline-recognition portion and thereby alter the fluorescence of the composition without displacing the fluorescent dye from the fluorescent module; and (e) quantitating the fluorescence of the composition in contact with the test solution, wherein a difference between the fluorescence quantitated in step (c) and step (e) indicates that the theophylline is present in the test solution.

According to the present invention a method of detecting flavine mononucleotide phosphate in a test solution comprising: (a) providing a composition comprising an oligonucleotide which comprises the following structure, wherein M is a malachite green dye:

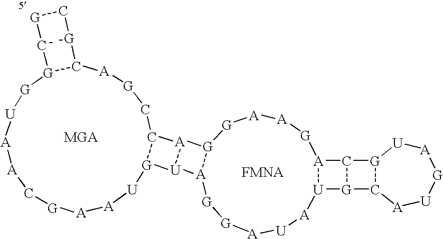

(b) contacting the composition with a control solution;(c) quantitating the fluorescence of the composition in contact with the control solution;(d) contacting the composition with the test solution under conditions which permit any of the flavine mononucleotide phosphate present in the test solution to bind to the flavine mononucleotide phosphate-recognition portion and thereby alter the fluorescence of the composition without displacing the fluorescent dye from the fluorescent module; and (e) quantitating the fluorescence of the composition in contact with the test solution, wherein a difference between the fluorescence quantitated in step (c) and step (e) indicates that the flavine mononucleotide phosphate is present in the test solution.

According to the present invention, a composition comprising an oligonucleotide is provided, comprising a recognition portion, a reporting portion, and a double stranded stem portion which connects the reporting portion to the recognition portion, wherein a fluorescent dye is bound to the reporting portion, and wherein the recognition portion is capable of binding a predetermined compound, and wherein the binding of the predetermined compound to the recognition portion alters the fluorescence of fluorescent dye bound to the reporting portion without displacing the fluorescent dye from the reporting portion.

According to the present invention a composition comprising the following structure:

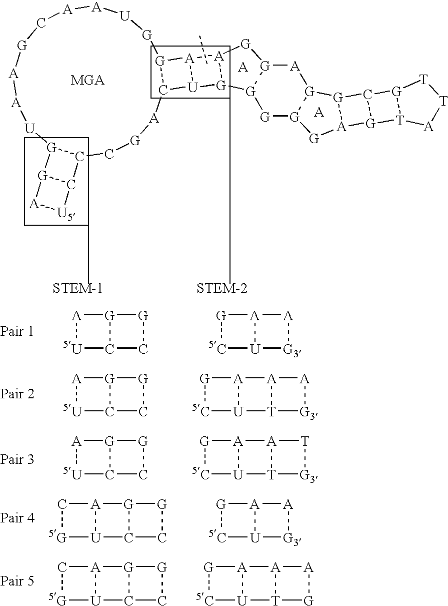

According to the present invention a composition is provided comprising the following structure:

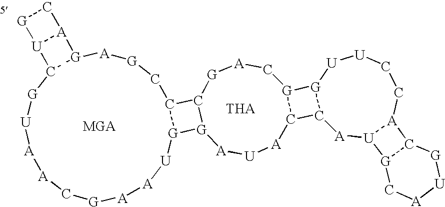

According to the present invention, a composition is provided comprising the following structure:

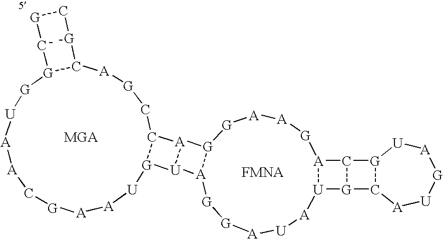

### BRIEF DESCRIPTION OF THE DRAWING

FIG. 1. A. Structure of malachite green (MG) and malachite green aptamer (MGA).

FIG. 2. A. Structures of seven sensors tested, with the results of the initial screening (F+/F− ratio of fluorescence intensities in the presence and absence of 1 mM ATP, FU absolute value of fluorescence intensity in the presence of ATP in relative fluorescence units.

FIG. 3. A. Fluorescence spectra of the MGA-ATPA. 1 in the presence of increasing amounts of ATP (from 2 mM serial dilutions, and no ATP, deep blue colored spectra); B. The matching fluorescence intensity (relative units FU) of the MGA-ATPA.1 in the presence of MG and increasing concentrations of ATP (diamonds), UTP (squares), CT (triangles) and GTP (crosses). Each spectra and data point represents the average of three consecutive scans.

FIG. 4. A. Structures of theophiline sensor (MGA-THA); B. Fluorescence intensity vs. concentration curves for theophiline (diamongs) and caffeine (squares). Each spectra and data point represents the average of three consecutive scans.

FIG. 5. Structure of FMN sensor (MGA-FMNA) and FMN.

FIG. 6. A. Fluorescence spectra increase in the presence of MG and increasing concentrations of FMN (background fluorescence is labeled red). B. The matching fluorescence intensity (relative fluorescence units) vs. concentration curves for flavin. Each spectra and data point represents the average of three consecutive scans.

### DETAILED DESCRIPTON OF THE INVENTION

According to the invention, a method of detecting whether a specific compound is present in a test solution is provided comprising: (a) providing a composition comprising an oligonucleotide comprising a recognition portion, a reporting portion, and a double stranded stem portion which connects the reporting portion to the recognition portion, wherein a fluorescent dye is bound to the reporting portion; (b) contacting the composition with a control solution; (c) quantitating the fluorescence of the composition in contact with the control solution; (d) contacting the composition with the test solution under conditions which permit any of the specific compound present in the test solution to bind to the recognition portion and thereby alter the fluorescence of the composition without displacing the fluorescent dye from the reporting portion; and (e) quantitating the fluorescence of the composition in contact with the test solution, wherein a difference between the fluorescence quantitated in step (c) and step (e) indicates that the specific compound is present in the test solution.

Any specific compound present in the test solution may increase the fluorescence of the composition, and will be detected when the fluorescence quantitated in step (c) is less than that quantitated in step (e).

Any specific compound present in the test solution may decrease the fluorescence of the composition, and will be detected when the fluorescence quantitated in step (c) is greater than that quantitated in step (e).

The control solution may be substantially free of the specific compound.

The control solution may contain a predetermined amount of the specific compound.

The specific compound may be ATP, theophylline, or flavine mononucleotide phosphate.

The fluorescent dye may be malachite green.

The recognition portion may comprise a circle of nucleotides.

The reporting portion may comprise a circle of nucleotides.

The composition further may comprise a second double stranded stem portion attached to the reporting portion but not attached to the recognition portion.

According to the invention, a method of detecting ATP in a test solution is provided comprising: (a) providing a composition comprising an oligonucleotide which comprises the following structure, wherein M is a malachite green dye: 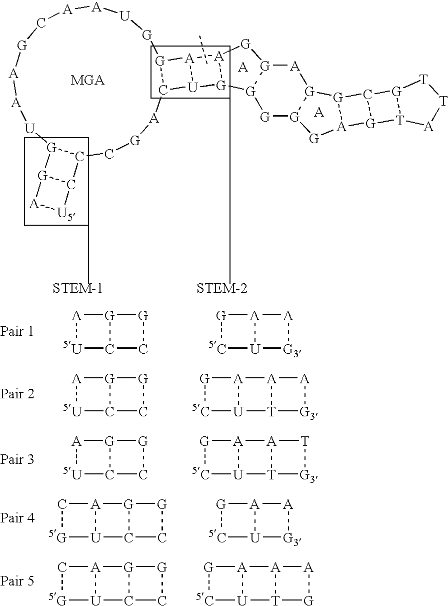
(b) contacting the composition with a control solution; (c) quantitating the fluorescence of the composition in contact with the control solution; (d) contacting the composition with the test solution under conditions which permit any of the ATP present in the test solution to bind to the ATP-recognition portion and thereby alter the fluorescence of the composition without displacing the fluorescent dye from the fluorescent module; and (e) quantitating the fluorescence of the composition in contact with the test solution,
wherein a difference between the fluorescence quantitated in step (c) and step (e) indicates that the ATP is present in the test solution.

According to the present invention a method of detecting theophylline in a test solution is provided comprising: (a)
providing a composition comprising an oligonucleotide which comprises the following structure, wherein M is a malachite green dye:

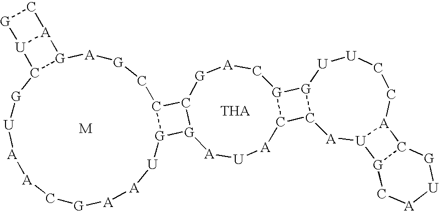

(b) contacting the composition with a control solution; (c) quantitating the fluorescence of the composition in contact with the control solution; (d) contacting the composition with the test solution under conditions which permit any of the theophylline present in the test solution to bind to the theophylline-recognition portion and thereby alter the fluorescence of the composition without displacing the fluorescent dye from the fluorescent module; and (e) quantitating the fluorescence of the composition in contact with the test solution, wherein a difference between the fluorescence quantitated in step (c) and step (e) indicates that the theophylline is present in the test solution.

According to the present invention a method of detecting flavine mononucleotide phosphate in a test solution comprising: (a) providing a composition comprising an oligonucleotide which comprises the following structure, wherein M is a malachite green dye:

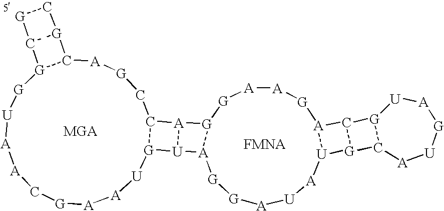

(b) contacting the composition with a control solution; (c) quantitating the fluorescence of the composition in contact with the control solution; (d) contacting the composition with the test solution under conditions which permit any of the flavine mononucleotide phosphate present in the test solution to bind to the flavine mononucleotide phosphate-recognition portion and thereby alter the fluorescence of the composition without displacing the fluorescent dye from the fluorescent module; and (e)quantitating the fluorescence of the composition in contact with the test solution, wherein a difference between the fluorescence quantitated in step (c) and step (e) indicates that the flavine mononucleotide phosphate is present in the test solution.

According to the present invention, a composition comprising an oligonucleotide is provided, comprising a recognition portion, a reporting portion, and a double stranded stem portion which connects the reporting portion to the recognition portion, wherein a fluorescent dye is bound to the reporting portion, and wherein the recognition portion is capable of binding a predetermined compound, and wherein the binding of the predetermined compound to the recognition portion alters the fluorescence of fluorescent dye bound to the reporting portion without displacing the fluorescent dye from the reporting portion.

According to the present invention a composition is provided comprising the following structure:

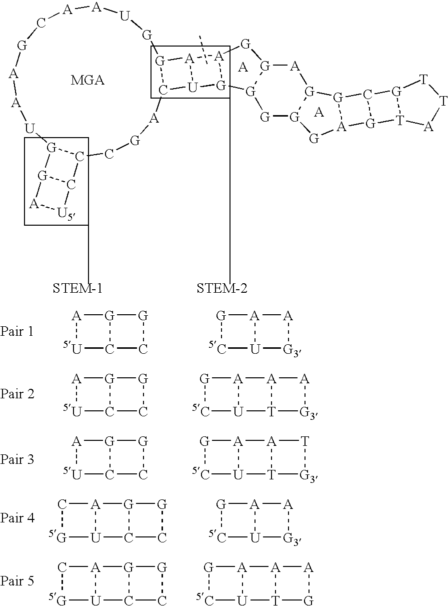

According to the present invention a composition is provided comprising the following structure:

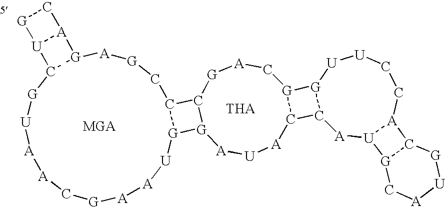

According to the present invention, a composition is provided comprising the following structure:

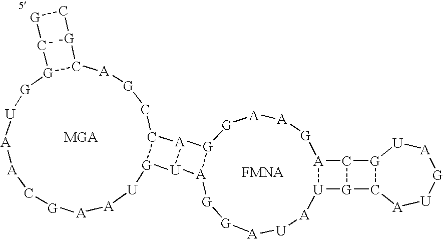

The term “recognition portion” as used herein means a part of an aptameric sensor comprising nucleotides configured so as to form a binding pocket able to bind the specific compound for which the aptamer is a sensor. The recognition portion is derived from traditional in vitro selection and amplification procedures.

The term “reporter portion” as used herein means a part of the modular aptameric sensor comprising nucleotides configured so as to form a binding pocket in which is bound a reporter moiety, such as a fluorophore. The binding of the reporter portion to the reporter moiety is regulated by the binding of the recognition portion to its compound.

The term “stem portion” as used herein means a double stranded (hybridized) section of an aptameric sensor configured so as to connect the reporter portion of the aptamer to the recognition portion of the aptamer.

The term “connecting stem portion” as used herein means a doubling (hybridized) section of an aptametric sensor configured so as to connect the reporter portion of the aptamer to the recognition portion of the aptamer. The connecting stem is not stable unless the recognition portion is complexed with the compound it recognizes.

Construction of chimeric ATP sensors: We used a chimeric construct, combining DNA aptamer binding ATP(18) (ATPA) with malachite green aptamer. This choice was the result of several considerations. First, this aptamer was successfully used in several approaches, (4,5) and we could clearly compare our approach to others. Next, this aptamer is comparably short, so we could rapidly have synthetic sensors assembled on an oligonucleotide synthesizer. Finally, the DNA part of the sensor guaranteed somewhat increased stability of the construct, at least toward endonucleases.

Design-wise, in an analogy to modularly designed nucleic acid catalysts (14-17), we expected that the typical modularly designed aptameric sensor would consist of three domains (modules): a signaling domain (malachite green aptamer), a recognition domain (analyte aptamer), and a connecting stem (communication module), which should tranduce the recognition of ATP into an increased recognition of malachite green, and, concomitantly increased fluorescence. The reported malachite green aptamer has two stems onto which another aptamer could be attached through a communication module. Again, in an analogy to nucleic acid catalysts, we decided to construct chimeras with signaling domain at the outer portion (5′ and 3′ ends) of the construct. Recognition and signaling domains have conserved core structures, so we focused our engineering efforts mostly on the communication module. We were particularly interested in achieving positive regulation, because any detection of an analyte is rendered more sensitive by the low background fluorescence in the absence of an analyte. Our idea was to connect two aptamers through their double helical regions, and then weaken the common stem until we see a response; which is defined as an increase in malachite green fluorescence upon increase in ATP concentration. In other words, we hoped to achieve the situation in which the binding of ATP would stabilize the formation of MG aptamer.

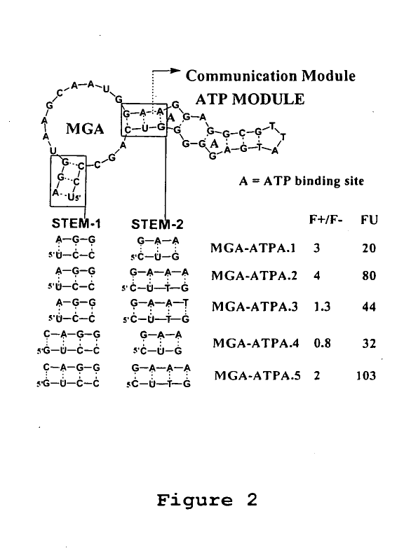

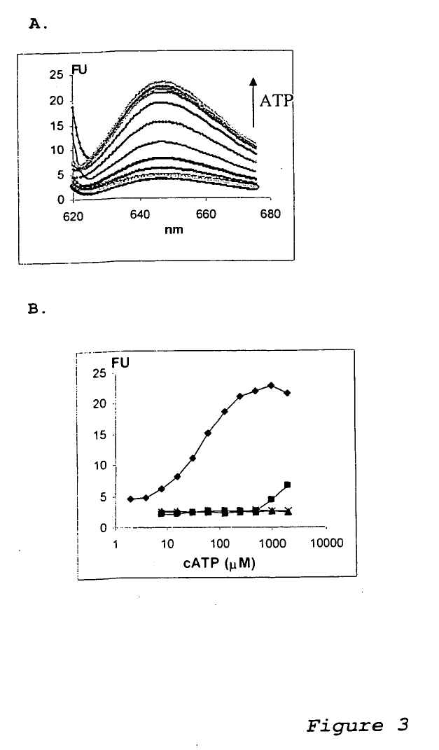

Initially, we constructed five chimeric constructs (FIG. 2) and tested them for fluorescence in the presence of 1 μM MG and in the buffer mimicking intracellular milieu (20 mM TRIS, pH=7.4, 140 mM KCl, 5 mM NaCl, 5 mM MgCl₂) in the presence and absence of 1 mM ATP. These chimeric candidates were constructed to address the influence of not only communication stem, but also an outer stem of the MGA. In general, what we observed is what could be explained by straightforward reasoning: (1) increased lengths of both outer and communication stems stabilized fluorescent complex formation, observed through an increase of fluorescence with and without ATP (MGA-ATPA.5), (2) decreased stability of the outer stem yielded greater difference in fluorescence with and without ATP (cf. MGA-ATPA.1 and MGA-ATPA.2), presumably because of the increased significance of the stabilization of communication stem; at the same time (3) mismatches close to the ATP binding sites (GA to GT mismatch, MGA-ATPA.4) diminish signaling. For further characterization we have chosen sensor MGA-ATPA.1, which showed both good response (almost three-fold increase), reasonable final fluorescence intensity and low background in the absence of ATP (FIG. 3A).

Characterization of MGA-ATPA.1 sensor: We first characterized the sensor over full range of concentrations of ATP, and for the selectivity over other NTP's. In a buffer mimicking intracellular milieu, with 1 μM sensor and 0.5 μM MG, the sensor responded over range of 10 μM-1 mM of ATP (FIG. 3A), with half-saturation (Kd apparent) at approximately 50 μM ATP, which is similar to other sensors based on this ATP aptamer. (3,5,9) The response was almost fivefold above background at the highest ATP concentrations, which is better than previously reported single molecule constructs (3), but less than the best heteromeric sensors (5) (“structure switching sensors”). This robustness of response is a promising characteristic for planned intracellular applications. An important distinction with previous sensors is that this is the first construct that does not require the covalent attachment of fluorophore for the sensor function. The selectivity of sensor closely followed the reported selectivity of the original aptamer. (2) Similar Kd change was confirmed by observing approximately three-fold shift in the concentration of aptamer-fluorescence signal curves for the increasing concentrations of sensor in the presence of saturating concentrations of ATP (2 mM) and in the absence of analyte with MG at 330 nM. Specifically, no response was observed with GTP and UTP, and only minimal response at the highest concentrations was observed with UTP (FIG. 3B).

To further characterize the MGA-ATPA.1, we attempted to saturate it (at 1 μM) with the excess of MG. However, at concentrations above 10 μM MG, fluorescence started decreasing, presumably due to non-specific interactions between the dye and the nucleic acid, possibly causing self-quenching. Based on comparison with the original aptamer (MGA), under the same conditions, we estimated that below 20% of the sensor is bound to malachite green at 10 μM MG. This indicates that Kd of the malachite green domain is around 40 μM. After the addition of the saturating concentrations of ATP, increased complex formation to about 40% was observed, indicating that in the presence of fully formed ATP binding pocket, the Kd of malachite green module drops to approximately 15 μM. (2) Similar Kd change was confirmed by observing approximately three-fold shift in the concentration of aptamer-fluorescence signal curves for the increasing concentrations of sensor in the presence of saturating concentrations of ATP (2 mM) and in the absence of analyte with MG at 330 nM. These results support our proposed mechanism of allosteric regulation of the binding strength of signaling module by recognition module.

Theophylline and FMN sensors: In order to demonstrate that our approach can be applied to the construction of other sensors for small molecules, we tested this design on two more analytes, theophylline (TH) and flavine mononucleotide phosphate (FMN).

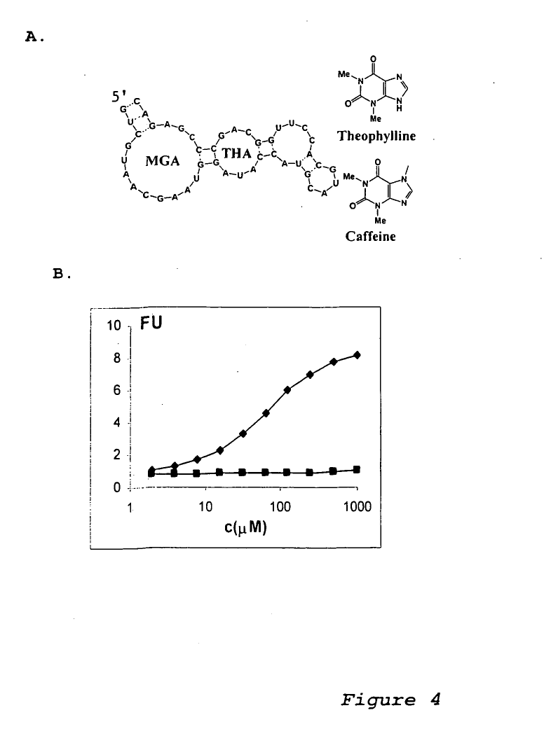

The theophyline aptamer (THA) (19) is unique for its ability to distinguish theophylline from the closely related caffeine with selectivity greater than any of the existing anti-theophylline antibodies (10). We were intrigued whether a modular sensor would be able to reproduce the exquisite selectivity of the parent aptamer. Accordingly, we constructed a theophyline sensor combining the malachite green aptamer with the theophylline aptamer through two Watson-Crick base pairs long stem to obtain MGA-THA, similar to the one used to construct MGA-ATPA.1 sensor. As expected, this construct, in the presence of 2 μM MG behaved as a sensor of theophylline with up to eightfold increase in fluorescence intensity over the TH range from 2-250 nM. Importantly, MGA-THA was completely insensitive to caffeine (FIG. 4B), which was in agreement with the supposition that aptamer-derived sensors conserve selectivities of their parent aptamers. Experiments similar to those performed for the MGA-ATPA.1 indicate that at the maximum concentration of MG (10 μM) using 1 μM aptamer, approximately 3% and 20% of signaling domains are formed in the absence of theophylline and in the presence of 1 mM theophylline, respectively, indicating a change in Kd for the signaling domain from approximately 300 μM to 50 μM with the ATP binding.

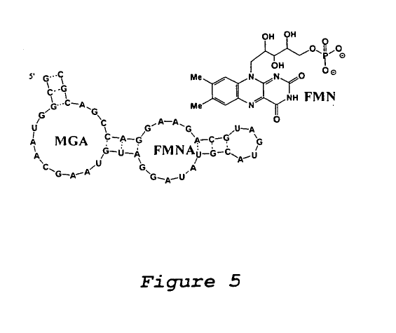

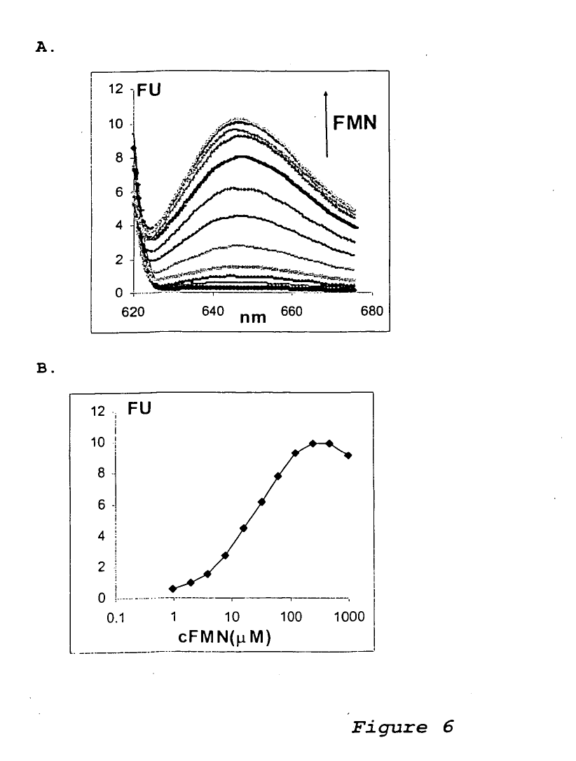

In our final construct, the FMN sensor MGA-FMA (FIG. 5) we decided to take the communication module reported to work for the catalyst switch. (14-17) Coincidentally, this module has again only two stable Watson-Crick base pairs. The communication modules in modular nucleic acid catalysts could have various lengths and the switching mechanisms may be different. While there was no a priori reason to assume that the mechanistic basis for the successful design of the catalytic nucleic acids would be translated into the success of the modular aptameric design, we were gratified to find out that the construct (FIG. 6) behaved as a sensor, with a 30-50-fold increase in fluorescence (or in binding of malachite green) at saturating concentrations of FMN. This impressive increase is based on the almost complete lack of binding to MG in the absence of theophiline, resulting in the low background fluorescence. The MGA-FMNA aptameric sensor, to the best of our knowledge, has the most robust signal of all reported aptameric sensors in the peer-reviewed literature. Using maximum concentrations of malachite green, the sensor is less than 1.5% bound to the dye, indicating a >750 μM Kd. The addition of FMN causes around 15% of the signaling module to form a complex, and the Kd shifted to approximately 30 μM. Importantly, omission of the aptamer yielded an essentially non-fluorescent solution.

The second aspect that would be critical for the widespread practical, that is, intracellular, applications is the choice of a chromophore. Whether malachite green is the best choice for intracellular signaling remains an open question, at least until details of the first mRNA intracellular tracking experiments using MG are published by Tsien's group. Namely, malachite green generates very efficiently singlet oxygen upon irradiation. This property was previously used to achieve targeted damage of mRNA constructs (12), and may lead to the undesired behavior of cells during imaging process and severe limitations in experimental set-ups. For example, even in vitro, we noticed that extensive irradiation leads to the reduction of fluorescent signal, and we attributed this property to the photodestruction of RNA. Fortunately, other dyes with potentially different photooxidation properties are also available for use in these constructs (11). We also note that our results with covalently attached fluorescein (6), together with earlier observations by Ellington's group (3), indicate that the construction of the binding pocket for fluorescein could lead to significant quenching in the bound state and robust signaling (for example up to four-fold increase in signal was observed in some of the sensors based on three-way junctions used in cross-reactive arrays). Finally, we note that it would be desirable to construct ratiometric sensors based on aptamers to provide internal control in the quantitative assessment of changes in the analyte concentrations.

One limitation in the rational modular design approach to sensors, described in this paper, is that some of the aptamers with preformed binding conformations lacking appropriate stems may be more difficult to use as recognition domains for the positively regulated allosteric sensors. For example, we were not able to achieve positive allosteric regulation by thrombin using reported G-quartet based aptamer (20), despite several tested chimeric constructs. However, we were able to use the steric bulk of the protein to achieve what we presume is negative steric regulation and release of malachite green. Also, we were not able to achieve an increase in fluorescence for the ATP aptamer that was previously reported to undergo steric clash with the ribozyme-domain in the allosterically regulated nucleic acid catalysts. Again, a selection process (that is, a non-rational, combinatorial process) could be used to rectify this weakness in rational approach, and we will describe our advances in this area in due course.

Through our previous work, we have introduced the principles of modular design into the molecular computation area. (21) Our successful construction of aptameric modular sensors expands the principles of modular design to yet another area, providing a new venue for the construction of molecular sensors. The robustness of responses and the fact that some of our sensors are made only of natural RNA components indicate that similar constructs have potential for applications in intracellular imaging.

Materials and Methods

Materials: Oligonucleotides were custom made and DNA/RNAse free HPLC purified by Integrated DNA Technologies Inc. (Coralville, Iowa) or TriLink Biotechnologies (San Diego, Calif.) and used as received. DNAse/RNAse free water was purchased from ICN (Costa Mesa, Calif.) and used for all buffers, and for stock solutions of sensors, which were made at 100 μM. NTP stock solutions (100 mM) were purchased from Promega (Madison, Wis.), malachite green, theophylline, caffeine and FMN were purchased from Sigma-Aldrich Co. (Milwaukee, Wis.). Binding buffer approximately mimicking intracellular milieu was used for all experiments (20 mM Tris, pH=7.4, 5 mM MgCl2, 140 mM KCl, 5 mM NaCl).

Instrumental: Fluorescent spectra were taken on a Perkin-Elmer (San Jose, Calif.) LS-55 Luminometer with Hamamatsu Xenon Lamp. Experiments were performed at the excitation wavelength of 610 nm and emission scan at 620-700 nm. The spectra were exported to Microsoft Excel files and colored appropriately.

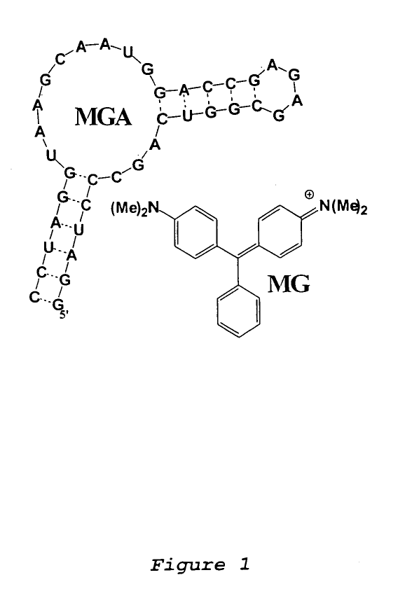

Characterization of MGA-ATPA.1 and MGA-FMNA: Sensors were diluted in binding buffer to 1 μM concentration, and malachite green (1 mM stock solution in water) was added at desired concentrations (e.g., in the experiments for FIG. 1, 0.5 μM). Series of standard dilutions of analytes (ATP, CTP, UTP, GTP, 100 mM stock, FM, 52 mM stock) were performed in sensor soluiton, and three fluorescent readings were taken with each solution within five minutes.

Characterization of MGA-THA: Sensor was diluted in binding buffer to 1 μM concentration, and malachite green added to 2 μM. To avoid dilution in the first samples (due to low solubility of theophylline and caffeine and a diluted stock solution of 1 mM), two-fold serial dilutions were performed in buffer, and sensor solution added afterwards.

Estimations of Kd's: Sensor was diluted in binding buffer to 1 μM concentration, and malachite green added to 10 μM. The fluorescence intensity of this sample was compared to the fluorescence intensity of the MGA aptamer under same conditions. This was used to estimate the % formed complex in the absence of ligand (calculated as the ratio of fluorescence intensities between solutions of sensors, and MGA solution), % free aptamer (nonfluorescent species, calculated based on the complex formation extent), and the concentration of malachite green (nonfluorescent species, in most of the cases assumed unchanged, due to the large excess of dye). These values were used directly in the equation for Kd to estimate its value. Exactly the same experiment was performed in the presence of the saturating concentrations of individual ligands.

Supporting Information: 1. Fluorescence values for experiments peformed in the presence of 10 μM MG: (a) for ATP; (b) for Theophylline; (c) for FMN. 2. Saturation of fluorescence in the presence of 10 μM MG exemplified on 1 μM MGA-THA sensor. 3. Concentration of aptamer-fluorescence signal curves for increasing concentrations of aptamer in the presence of 330 nM MG and presence and absence of 1 mM ATP.

### REFERENCES

1) Jayasena, S. D. Clinical Chem. 1999, 45, 1628.
2) Yamamoto, R.; Kumar, P. K. R. Gen. Cell. 2000, 5, 389.
3) Jhaveri, S.; Rajendran, M.; Ellington, A. D. Nat. Biotechol. 2000, 18(12) 1293-1297.
4) Jhaveri, S. D.; Kirby, R.; Conrad, R.; Maglott, E. J.; Bowser, M.; Kennedy, R. T; Glick, G.; Ellington, A. D. J. Am. Chem. Soc. 2000, 122, 2469-2473.
5) Nutiu, R., Li, Y. J. Am. Chem. Soc. 2003, 125, 4771-4778.
6) Stojanovic, M. N.; Green, E. G.; Semova, S.; Landry, D. W. J. Am. Chem. Soc. 2003, 125, 6085-6089.
7) Stojanovic, M. N.; Landry, D. W. J. Am. Chem. Soc. 2002, 124, 9678-9679.
8) Stojanovic, M. N.; de Prada, P.; Landry, D. W. J. Am. Chem. Soc. 2000, 122, 11547-11548;
9) Stojanovic, M. N.; de Prada, P.; Landry, D. W. J. Am. Chem. Soc. 2001, 123, 4503-4508.
10) Zhang, J.; Campbell, R. E.; Ting, A. Y.; Tsien, R. Y. Nature Rev. Mol. Cell Biol. 2002, 3, 906.
11) Grate, D.; Wilson, C. Proc. Natl. Acad. Sci. U. S. A. 1999, 96, 6131-6136.
12) Babendure, J. R.; Adams, S. R.; Tsien, R. Y. J. Am. Chem. Soc. 2003, 125, 14716.
13) Cholic acid competes for binding with Cibacron Blue in allosteric aptamer: Wu, L.; Curran, J. F. Nucleic Acid Res. 1999, 27, 1512.
14) Breaker R. R. Current Opinion in Biotechnology 2002, 13, 31-39.
15) Tang, J.; Breaker, R. R. Nuceic Acid Res. 1998, 26, 4214-4221.
16) Soukup, G.; Breaker, R. R. Proc. Nat. Acad. Sci. U. S. A. 1999, 96, 3584-3589.
17) Robertson, M. P.; Ellington, A. D. Nucleic Acids Res. 2000, 28, 1751-1759.
18) Sasanfar, M.; Szostack, J. W. Nature 1993, 364, 550-553.
19) Jenison, R. D., Gill, C., Pardi, A., Polisky, B. Science, 1994, 263, 1425-9.
20) Bock, L. C.; Griffin, L. C.; Latham, J. A.; Vermaas, E. H.; Toole, J. J. Nature 1992, 355, 564-566.
21) Stojanovic, M. N., Mitchell, T. E. & Stefanovic, D. Deoxyribozyme-based Logic Gates. J. Am. Chem. Soc. 124, 123-125 (2002).

I claim:

1. A method of detecting whether a specific compound is present in a test solution comprising:
(a) providing a composition comprising an oligonucleotide comprising a recognition portion, a reporting portion, and a double stranded stem portion which connects the reporting portion to the recognition portion, wherein a fluorescent dye is bound to the reporting portion;
(b) contacting the composition with a control solution;
(c) quantitating the fluorescence of the composition in contact with the control solution;
(d) contacting the composition with the test solution under conditions which permit any of the specific compound present in the test solution to bind to the recognition portion and thereby alter the fluorescence of the composition without displacing the fluorescent dye from the reporting portion; and
(e) quantitating the fluorescence of the composition in contact with the test solution,
wherein a difference between the fluorescence quantitated in step (c) and step (e) indicates that the specific compound is present in the test solution.

2. The method of claim 1, wherein any specific compound present in the test solution will increase the fluorescence of the composition, and will be detected when the fluorescence quantitated in step (c) is less than that quantitated in step (e).

3. The method of claim 1, wherein any specific compound present in the test solution will decrease the fluorescence of the composition, and will be detected when the fluorescence quantitated in step (c) is greater than that quantitated in step (e).

4. The method of claim 1, wherein the control solution is substantially free of the specific compound.

5. The method of claim 1, wherein the control solution contains a predetermined amount of the specific compound.

6. The method of claim 1, wherein the specific compound is ATP, theophylline, or flavine mononucleotide phosphate.

7. The method of claim 1, wherein the fluorescent dye is malachite green.

8. The method of claim 1, wherein the recognition portion comprises a circle of nucleotides.

9. The method of claim 1, wherein the composition further comprises a second double stranded stem portion attached to the reporting portion but not attached to the recognition portion.

10. A method of detecting ATP in a test solution comprising:
(a) providing a composition comprising an oligonucleotide which comprises the following structure, wherein MGA is a malachite green dye and A is an ATP binding site;
and wherein stem 1 and stem 2 are chosen from the pairs shown below: 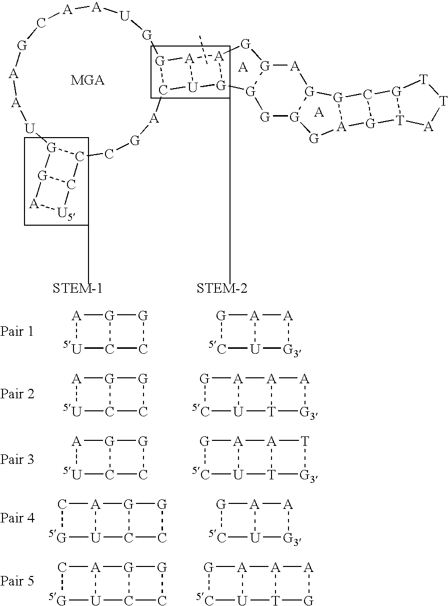
(b) contacting the composition with a control solution;
(c) quantitating the fluorescence of the composition in contact with the control solution;
(d) contacting the composition with the test solution under conditions which permit any of the ATP present in the test solution to bind to the ATP-recognition portion and thereby alter the fluorescence of the composition without displacing the fluorescent dye from the fluorescent module; and
(e) quantitating the fluorescence of the composition in contact with the test solution,
wherein a difference between the fluorescence quantitated in step (c) and step (e) indicates that the ATP is present in the test solution.

11. A method of detecting theophylline in a test solution comprising:
(a) providing a composition comprising an oligonucleotide which comprises the following structure, wherein MGA is a malachite green dye and THA is a theophylline binding site: 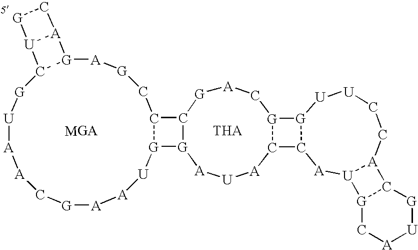
(b) contacting the composition with a control solution;
(c) quantitating the fluorescence of the composition in contact with the control solution;
(d) contacting the composition with the test solution under conditions which permit any of the theophylline present in the test solution to bind to. the theophylline-recognition portion and thereby alter the fluorescence of the composition without displacing the fluorescent dye from the fluorescent module; and
(e) quantitating the fluorescence of the composition in contact with the test solution,
wherein a difference between the fluorescence quantitated in step (c) and step (e) indicates that the theophylline is present in the test solution.

12. A method of detecting flavine mononucleotide phosphate in a test solution comprising:
(a) providing a composition comprising an oligonucleotide which comprises the following structure, wherein MGA is a malachite green dye and FMNA is a flavine mononucledide phosphate binding site: 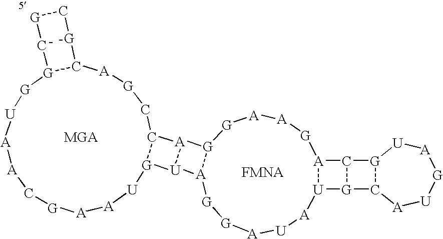
(b) contacting the composition with a control solution;
(c) quantitating the fluorescence of the composition in contact with the control solution;
(d) contacting the composition with the test solution under conditions which permit any of the flavine mononucleotide phosphate present in the test solution to bind to the flavine mononucleotide phosphate-recognition portion and thereby alter the fluorescence of the composition without displacing the fluorescent dye from the fluorescent module; and
(e) quantitating the fluorescence of the composition in contact with the test solution,
wherein a difference between the fluorescence quantitated in step (c) and step (e) indicates that the flavine mononucleotide phosphate is present in the test solution.

13. A composition comprising an oligonucleotide comprising a recognition portion, a reporting portion, and a double stranded stem portion which connects the reporting portion to the recognition portion, wherein a fluorescent dye is bound to the reporting portion, and wherein the recognition portion is capable of binding a predetermined compound, and wherein the binding of the predetermined compound to the recognition portion alters the fluorescence of fluorescent dye bound to the reporting portion without displacing the fluorescent dye from the reporting portion.

14. A composition comprising the following structure: wherein MGA is a malachite green dye and a is an ATP binding site; and wherein stem 1 and stem 2 are chosen from the pairs shown below:

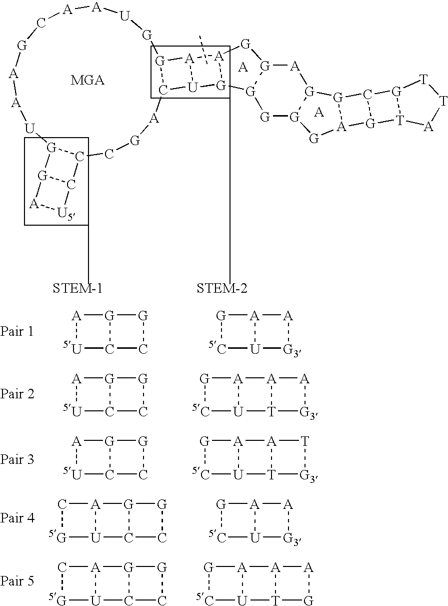

15. A composition comprising the following structure wherein MGA is a malachite green dye and THA is a theophylline binding site.

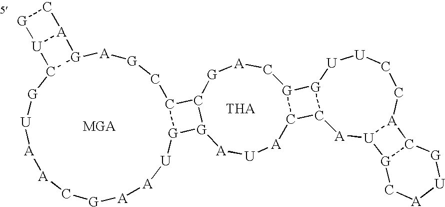

16. A composition comprising the following structure where MGA is a malachite green dye and FMNA is a flavine mononucleotide phosphate binding site: [chemical structure]
